SEQUENCE LISTING (20 sequences)

SEQ ID NO: 1 - DNA, 15 bp, Mus musculus
gacacctatatgcac

SEQ ID NO: 2 - DNA, 51 bp, Mus musculus
tggattgatcctgcgaatggaaataccaaatatgacccgaagttccagggc

SEQ ID NO: 3 - DNA, 30 bp, Mus musculus
tctggggttagtacggcccactttgactac

SEQ ID NO: 4 - DNA, 30 bp, Mus musculus
agggccagctcaagtgtaagtttcatgcac

SEQ ID NO: 5 - DNA, 21 bp, Mus musculus
gccacatccaacctggcttct

SEQ ID NO: 6 - DNA, 27 bp, Mus musculus
cagcagtggagtagttacccacggacg

SEQ ID NO: 7 - protein, 5 aa, Mus musculus
DTYMH

SEQ ID NO: 8 - protein, 17 aa, Mus musculus
WIDPANGNTKYDPKFQG

SEQ ID NO: 9 - protein, 10 aa, Mus musculus
SGVSTAHFDY

SEQ ID NO: 10 - protein, 10 aa, Mus musculus
RASSSVSFMH

SEQ ID NO: 11 - protein, 7 aa, Mus musculus
ATSNLAS

SEQ ID NO: 12 - protein, 7 aa, Mus musculus
ATSNLAS

SEQ ID NO: 13 - DNA, 414 bp, Mus musculus
atgaaatgcagctgggttatcttcttcctgatggcagtggttacaggggtcaattcagaggttcagctgcagcagtctgggacagaacttgtgaagccaggggcctcagtcaagttgtcctgcacaacttctggcttcaacattcaagacacctatatgcactgggtgaagcagaggcctgaacagggcctggagtggattggatggattgatcctgcgaatggaaataccaaatatgacccgaagttccagggcaaggccactataatagcagacacatcctccaacacagcctacctgcagctccgcggcctgacatctgaggacactgccgtctattactgtgctagatctggggttagtacggcccactttgactactggggccaaggcaccactctcacagtctcctca

SEQ ID NO: 14 - protein, 138 aa, Mus musculus
MKCSWVIFFLMAVVTGVNSEVQLQQSGTELVKPGASVKLSCTTSGFNIQDTYMHWVKQRPEQGLEWIGWIDPANGNTKYDPKFQGKATIIADTSSNTAYLQLRGLTSEDTAVYYCARSGVSTAHFDYWGQGTTLTVSS

SEQ ID NO: 15 - DNA, 384 bp, Mus musculus
atggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagtttcatgcactggtaccagcagaagccaggatcctcccccaaaccctggatttatgccacatccaacctggcttctggagtccctactcgcttcagtggcagtgggtctgggacctcttactctctcacactcagcagagtggaggctgaagatgctgccacttattactgccagcagtggagtagttacccacggacgttcggtggaggcaccaaactggaaatcaaa

SEQ ID NO: 16 - protein, 128 aa, Mus musculus
MDFQVQIFSFLLISASVIMSRGQIVLSQSPAILSASPGEKVTMTCRASSSVSFMHWYQQKPGSSPKPWIYATSNLASGVPTRFSGSGSGTSYSLTLSRVEAEDAATYYCQQWSSYPRTFGGGTKLEIK

SEQ ID NO: 17 - DNA, 357 bp, Mus musculus
gaggttcagctgcagcagtctgggacagaacttgtgaagccaggggcctcagtcaagttgtcctgcacaacttctggcttcaacattcaagacacctatatgcactgggtgaagcagaggcctgaacagggcctggagtggattggatggattgatcctgcgaatggaaataccaaatatgacccgaagttccagggcaaggccactataatagcagacacatcctccaacacagcctacctgcagctccgcggcctgacatctgaggacactgccgtctattactgtgctagatctggggttagtacggcccactttgactactggggccaaggcaccactctcacagtctcctca

SEQ ID NO: 18 - protein, 119 aa, Mus musculus
EVQLQQSGTELVKPGASVKLSCTTSGFNIQDTYMHWVKQRPEQGLEWIGWIDPANGNTKYDPKFQGKATIIADTSSNTAYLQLRGLTSEDTAVYYCARSGVSTAHFDYWGQGTTLTVSS

SEQ ID NO: 19 - DNA, 318 bp, Mus musculus
caaattgttctctcccagtctccagcaatcctgtctgcatctccaggggagaaggtcacaatgacttgcagggccagctcaagtgtaagtttcatgcactggtaccagcagaagccaggatcctcccccaaaccctggatttatgccacatccaacctggcttctggagtccctactcgcttcagtggcagtgggtctgggacctcttactctctcacactcagcagagtggaggctgaagatgctgccacttattactgccagcagtggagtagttacccacggacgttcggtggaggcaccaaactggaaatcaaa

SEQ ID NO: 20 - protein, 106 aa, Mus musculus
QIVLSQSPAILSASPGEKVTMTCRASSSVSFMHWYQQKPGSSPKPWIYATSNLASGVPTRFSGSGSGTSYSLTLSRVEAEDAATYYCQQWSSYPRTFGGGTKLEIK

(19) United States
(12) Patent Application Publication
(10) Pub. No.: US 2018/0064825 A1
(43) Pub. Date: Mar. 8, 2018

(54) ANTI-PD-L1 IMMUNOTOXIN FOR USE IN THERAPY

(71) Applicants: INSERM (INSTITUT NATIONAL DE LA SANTE ET DE LA RECHERCHE MEDICALE), Paris (FR); UNIVERSITE D'AIX-MARSEILLE, Marseille (FR); INSTITUT JEAN PAOLI & IRENE CALMETTES, Marseille (FR); CENTRE NATIONAL DE LA RECHERCHE SCIENTIFIQUE - CNRS, Paris (FR)

(72) Inventor: Daniel OLIVE, Marseille (FR)

(21) Appl. No.: 15/563,324

(22) PCT Filed: Apr. 6, 2016
(86) PCT No.: PCT/EP2016/057476

§ 371 Date: Sep. 29, 2017

(30) Foreign Application Priority Data
Apr. 7, 2015 (EP) 15305511.6

(51) Int. Cl. A61K 47/68; C07K 16/28; C07K 16/30; A61K 51/10

(52) U.S. Cl. CPC A61K 47/6851; C07K 16/2827; C07K 16/30; A61K 2039/505; A61K 47/6817; A61K 47/6803; A61K 51/1096

(57) ABSTRACT

The present disclosure relates to a combination of an anti-PD-L1 (Programmed Cell Death 1 Ligand 1) with an ‘immunotoxin’ for use in a method for treating a tumor in a patient. Experimental results are provided for a combination of an anti POL 1 antibody with the radioisotope Indium 111.

### FIELD OF THE INVENTION

The present invention relates to a method for treating patients suffering from or suspected to suffer from a cancer involving a solid tumor.

### BACKGROUND OF THE INVENTION

Treatment of solid tumors is a major concern of public health. Over the past 30 years, fundamental advances in the chemotherapy of neoplastic diseases have been realized. However, despite the impressive advances that have been made, many of the most prevalent forms of human cancers, for example, solid tumors such as prostate, breast or pancreatic cancer are still difficult to treat efficiently. One major limitation of the prior art is the lack of efficient methods for identifying patients suffering or at risk of suffering from said disease, and sorting out patient who might benefit from specific therapeutic strategies. The quest for prognosis biomarkers is thus ongoing challenge.

Thus, there is a need for identification of diagnostic molecules that would provide an improvement over existing biomarkers for patient suspected of suffering from solid tumors.

In addition, the currently available therapeutic strategy are often ineffective against these tumors due, in large part, to the difficulty in achieving therapeutically effective levels of chemotherapeutic agents in the area of tumor growth and infiltration. Even solid tumor therapies, which are not restricted by the existence of the blood brain barrier, can give unsatisfactory results. For example, prostate cancer is a common form of cancer among males, and there are cases of aggressive prostate cancers. Clinical evidence shows that human prostate cancer has the propensity to metastasize to bone and lymph nodes and is currently in the USA the second leading cause of cancer death, after lung cancer, among men. Commonly, treatment is based on surgery and/or radiation therapy and/or chemotherapy, but these methods give unsatisfactory results in a significant percentage of cases. Pancreatic cancer is another example of solid tumor. It has one of the highest mortality rates of any malignancy, and it is the fourth most common cause of cancer-related deaths in the USA. The poor prognosis of this malignancy is a result of the difficulty of early diagnosis and poor response to current therapeutic methods.

Thus, it is clear that there is a need for improving the currently available therapies for the treatment of cancers involving solid tumors and/or for developing new therapies targeting said solid tumors.

### SUMMARY OF THE INVENTION

The inventors have shown that it is possible to efficiently target PD-L1-positive tumors using radiolabelled anti-PD-L1 antibodies, providing a new treatment for patients suffering from PD-L1-expressing tumors.

Accordingly, the present invention relates to an anti-PD-L1 immunotoxin for use in a method of treating a tumor, preferably a PD-L1-positive tumor in a patient.

The invention also relates to a method for treating a patient suffering from a tumor, preferably a PD-L1-postive tumor, comprising the step of administering to said patient a therapeutically effective amount of anti-PD-L1 immunotoxin.

### DETAILED DESCRIPTION OF THE INVENTION

T cells play an essential role in the anti-cancer immune response. T cell activation depends on the initial antigen-specific signal, presented via the antigen-loaded major histocompatibility complex (MHC) to the T cell receptor, and on activation of the costimulatory molecule CD28 by binding of CD80/86. T cells also express coinhibitory molecules that are capable of downregulating the immune response (1). One major coinhibitory receptor is programmed death 1 (PD-1). PD-1 has two ligands, programmed death ligand-1 (PD-L1) and PD-L2, of which PD-L1 is most widely expressed. Binding of PD-L1 to PD-1 transduces an inhibitory signal to the T cell, resulting in inhibition of T cell proliferation, reduced secretion of effector cytokines, and potentially exhaustion. By up-regulating PD-L1 expression levels, tumor cells are capable of escaping immune recognition and attack (2-4).

PD-L1 is expressed on a wide variety of tumors, including breast cancer, gastric cancer, renal cell cancer, ovarian cancer, non-small lung cancer, melanoma, and hematological cancers (5). In general, PD-1 and PD-L1 have been demonstrated to be poor prognostic factors as high expression levels are associated with poor outcome of cancer patients (5). Preclinical studies with anti-PD-1 and anti-PD-L1 antibodies have shown promising anti-tumor effects and have led to the initiation of several clinical investigations. Early clinical trials demonstrated objective and durable (≧1 year) responses in patients with treatment-refractory, advanced melanoma, renal cell carcinoma, non-small cell lung cancer, and ovarian cancer (6-11). Because of these impressive results, phase II/III studies are currently further exploring the therapeutic efficacy of these agents. Due to the impressive efficacy in melanoma patients, the FDA has recently granted accelerated approval of pembrolizumab (anti-PD-1 antibody) for the treatment of patients with advanced or unresectable melanoma following progression on prior therapies (11).

As explained above, the inventors have shown that labelled anti-PD-L1 antibodies are targeted specifically to tumors expressing PD-L1. Anti-PD-L1 antibodies coupled to a toxin can therefore be used in therapy in order to target and kill tumor cells.

Thus, the present invention relates to an anti-PD-L1 immunotoxin for use in a method of treating a tumor, preferably a PD-L1-positive tumor, in a patient.

PD-L1, also known as CD274, Programmed Cell Death 1 Ligand 1 (PDCD1LG1 or PD-L1) or B7-H1, is a type I transmembrane glycoprotein composed of IgC- and IgV-type extracellular domains, which binds to PD1.

The terms “PD1”, “PD-1” and “Programmed cell death protein 1” refer to a member of the CD28 superfamily that delivers negative signals upon interaction with its two ligands, PD-L1 or PD-L2. PD-1 and its ligands are broadly expressed and exert a wider range of immunoregulatory roles in T cells activation and tolerance compared with other CD28 members. PD-1 was isolated as a gene up-regulated in a T cell hybridoma undergoing apoptosis and was named program death 1.

As used herein, the term ‘immunotoxin” has its general meaning in the art. By “immunotoxin”, it is meant a chimeric protein made of an antibody or modified antibody or antibody fragment (also called in the present application “antibody”), attached to a fragment of a toxin. The antibody of the immunotoxin is covalently attached to the fragment of a toxin. Preferably, the fragment of the toxin is linked by a linker to the antibody or fragment thereof. Said linker is preferably chosen from 4-mercaptovaleric acid and 6-maleimidocaproic acid.

The term “anti-PD-L1 immunotoxin” refers to an antibody-drug conjugate (ADC) wherein the antibody moiety is an anti-PD-L1 antibody and wherein said anti-PD-L1 antibody is linked to a toxin.

Upon binding to PD-L1 on its target cells, the immunotoxin enters the cells and kills the target cells.

As used herein, the term “antibody” has its general meaning in the art. The term “anti-PD-L1 antibody” refers to an antibody that binds specifically to PD-L1. Preferably, said antibody does not bind to PD-L2.

Antibodies specifically directed against PD-L1 may be derived from a number of species including, but not limited to, rodent (mouse, rat, rabbit, guinea pig, hamster, and the like), porcine, bovine, equine or primate and the like. Antibodies from primate (monkey, baboon, chimpanzee, etc.) origin have the highest degree of similarity to human sequences and are therefore expected to be less immunogenic. Antibodies derived from various species can be “humanized” by modifying the amino acid sequences of the antibodies while retaining their ability to bind the desired antigen. Antibodies may also be derived from transgenic animals, including mice, which have been genetically modified with the human immunoglobulin locus to express human antibodies. Procedures for raising “polyclonal antibodies” are well known in the art. For example, polyclonal antibodies can be obtained from serum of an animal immunized against PD-L1, which may be produced by genetic engineering for example according to standard methods well-known by one skilled in the art. Typically, such antibodies can be raised by administering PD-L1 protein subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times at six weeks' interval. A sample of serum is then collected 10 days after each boost. Polyclonal antibodies are then recovered from the serum by affinity chromatography using the corresponding antigen to capture the antibody. This and other procedures for raising polyclonal antibodies are disclosed by (Harlow et al., 1988), which is hereby incorporated in the references.

Although historically monoclonal antibodies were produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988).

A “monoclonal antibody” or “mAb” in its various names refers to a population of antibody molecules that contains only one species of antibody combining site capable of immunoreacting with a particular epitope. A monoclonal antibody thus typically displays a single binding affinity for any epitope with which it immunoreacts. Monoclonal antibody may also define an antibody molecule which has a plurality of antibody combining sites, each immunospecific for a different epitope. For example, a bispecific antibody would have two antigen binding sites, each recognizing a different interacting molecule, or a different epitope. As used herein, the terms “antibody fragment”, “antibody portion”, “antibody variant” and the like include any protein or polypeptide containing molecule that comprises at least a portion of an immunoglobulin molecule such as to permit specific interaction between said molecule and an antigen (e.g. PD-L1). The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of a ligand or counter-receptor which can be incorporated into an antibody of the present invention to permit interaction with the antigen.

Monoclonal antibodies (mAbs) may be prepared by immunizing a mammal such as mouse, rat, primate and the like, with purified PD-L1 protein. The antibody-producing cells from the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in (Kohler and Milstein, 1975). Alternatively, the immunoglobulin genes may be isolated and used to prepare a library for screening for reactive specifically reactive antibodies. Many such techniques including recombinant phage and other expression libraries are known to one skilled in the art.

While mAbs can be produced by hybridoma culture the invention is not to be so limited. Also contemplated is the use of mAbs produced by cloning and transferring the nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

In a particular embodiment, mAbs recognizing PD-L1 may be generated by immunization of Balb-c mice with the respective recombinant human Fc-IgG1 fusion proteins. Spleen cells were fused with X-63 myeloma cells and cloned according to already described procedures (Olive D, 1986). Hybridoma supernatants were then screened by staining of transfected cells and for lack of reactivity with untransfected cells.

Antibody generation techniques not involving immunisation are also contemplated such as for example using phage display technology to examine naive libraries (from non-immunised animals); see (Barbas et al., 1992, and Waterhouse et al. (1993). Antibodies of the invention are suitably separated from the culture medium by conventional immunoglobulin purification procedures such as, for example, affinity, ion exchange and/or size exclusion chromatography, and the like.

In a particular embodiment, the antibody of the invention may be a human chimeric antibody. Said human chimeric antibody of the present invention can be produced by obtaining nucleic sequences encoding VL and VH domains, constructing a human chimeric antibody expression vector by inserting them into an expression vector for animal cell having genes encoding human antibody CH and human antibody CL, and expressing the expression vector by introducing it into an animal cell. The CH domain of a human chimeric antibody may be any region which belongs to human immunoglobulin, but those of IgG class are suitable and any one of subclasses belonging to IgG class, such as IgG1, IgG2, IgG3 and IgG4, can also be used. Also, the CL of a human chimeric antibody may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. No. 5,202,238; and U.S. Pat. No. 5,204,244).

In another particular embodiment, said antibody may be a humanized antibody. Said humanized antibody may be produced by obtaining nucleic acid sequences encoding for CDRs domain by inserting them into an expression vector for animal cell having genes encoding a heavy chain constant region identical to that of a human antibody; and a light chain constant region identical to that of a human antibody, and expressing the expression vector by introducing it into an animal cell. The humanized antibody expression vector may be either of a type in which a gene encoding an antibody heavy chain and a gene encoding an antibody light chain exist on separate vectors or of a type in which both genes exist on the same vector (tandem type). In respect of easiness of construction of a humanized antibody expression vector, easiness of introduction into animal cells, and balance between the expression levels of antibody H and L chains in animal cells, a tandem type of the humanized antibody expression vector is more preferable (Shitara K et al. 1994). Examples of the tandem type humanized antibody expression vector include pKANTEX93 (WO 97/10354), pEE18 and the like. Methods for producing humanized antibodies based on conventional recombinant DNA and gene transfection techniques are well known in the art (See, e.g. Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576). Preferably, the anti-PD-L1 antibody fragments are chosen from Fab (e.g., by papain digestion), Fab′ (e.g., by pepsin digestion and partial reduction), F(ab)2, F(ab′)2 (e.g., by pepsin digestion) and dAb fragments.

Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Said Fab fragment of the present invention can be obtained by treating an antibody which specifically reacts with human PD-L1 with a protease, papaine. Also, the Fab may be produced by inserting DNA encoding Fab of the antibody into a vector for prokaryotic expression system or for eukaryotic expression system, and introducing the vector into a prokaryote or eucaryote to express the Fab.

Said F(ab′)₂ of the present invention may be obtained by treating an antibody which specifically reacts with PD-1 with a protease, pepsin. Also, the F(ab′)₂ can be produced by binding Fab′ described below via a thioether bond or a disulfide bond.

Said Fab′ may be obtained by treating F(ab′)₂ which specifically reacts with PD-1 with a reducing agent, dithiothreitol. Also, the Fab′ can be produced by inserting DNA encoding Fab′ fragment of the antibody into an expression vector for prokaryote or an expression vector for eukaryote, and introducing the vector into a prokaryote or eukaryote to effect its expression.

Preferably, the anti-PD-L1 antibody derivatives are chosen from scFv, (scFv)2, diabodies, multimeric scFv derived from an anti-PD-L1 antibody and fused to a Fc fragment, whole anti-PD-L1 antibodies linked together to reach an aggregated form, and antibodies containing at least two Fabs bound face-to-tail.

Said scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. No. 5,859,205; U.S. Pat. No. 5,585,089; U.S. Pat. No. 4,816,567; EP0173494).

In a preferred embodiment, the anti PD-L1 antibody according to the invention is a monoclonal antibody.

Several monoclonal anti-PD-L1 antibodies have been developed in the art and are available to the skilled artisan.

Suitable monoclonal anti-PD-L1 antibodies that can be used to produce an anti-PD-L1 immunotoxin according to the present invention, include, but are not limited to the monoclonal antibodies described and characterized in Ghiotto M, Gauthier L, Serriari N, Pastor S, Truneh A, Nunes J A, et al. PD-L1 and PD-L2 differ in their molecular mechanisms of interaction with PD-1. International immunology. 2010; 22:651-60, namely:
the PD-L1.1 antibody, obtainable from hybridoma deposited at the Collection Nationale de Cultures de Microorganismes (CNCM, Institut Pasteur, 25 rue du Docteur Roux, 75724 Paris Cedex 15, France), in accordance with the terms of Budapest Treaty, on Oct. 15, 2008 under deposited number CNCM 1-4080.
the PD-L1.2 antibody, obtainable from hybridoma deposited at the

Collection Nationale de Cultures de Microorganismes (CNCM, Institut Pasteur, 25 rue du Docteur Roux, 75724 Paris Cedex 15, France), in accordance with the terms of Budapest Treaty, on Oct. 15, 2008 under deposited number CNCM 1-4081.
the PD-L1.3 antibody, also called PD-L1.3.1 antibody, having the CDR sequences as described below.

Advantageously, the anti-PD-L1 antibody of the present invention has the following characteristics:
immunoreactivity is retained after radio labeling,
affinity for PD-L1 is high (typically, a Kd value of at least 10 nM, preferably a Kd value around 1.0 nM).
the antibody is internalized upon binding of the antibody to the tumor cell.
   The toxin (e.g. ¹¹¹In-DTPA) is therefore trapped in the tumor cell after internalization and degradation of the antibody.

In a particular embodiment, the anti-PD-L1 antibody used to produce the immunotoxin of the invention is an antibody that is internalized by tumor cells.

Internalization can be assessed according to any suitable method: flow cytometry (loss of PD-L1 expression), microscopy or western blot (protein disappearance. Typically, an anti-PD-L1 antibody is deemed to be internalized by tumor cells if it leads to cell death according to the in vitro test A described below.

Test A (internalization of the anti-PD-L1 antibody): Briefly, the prostate cell line PC3 is incubated with the anti-PD-L1 antibody with a range from 25 nM to 0.01 nM and a saporin-conjugated goat anti-mouse antibody IgG secondary antibody (referred to as Mab-ZAP, 50 ng) or a negative control saporin-conjugated pre-immune goat IgG (Ig-SAP, 50 ng) for 48 hours at 37° C. The anti-PD-L1 antibody/saporin complex is bound by the targeted cells positive for PD-L1 expression, internalized, and saporin is released to inactivate ribosomes.

Cell death is then measured by any suitable method. Typically, cell death can be measured by measuring caspase activity using the Promega caspase Glow 3/7 assay luminescence kit.

In one embodiment the anti-PD-L1 antibody according to the present invention is selected in the group consisting of the monoclonal antibody PD-L1.3.1, antibodies having the same CDRs as PD-L1.3.1, fragments and derivatives thereof having the above mentioned characteristics.

As used herein, the terms “PD-L1.3.1” and “PD-L1.3” are used interchangeably and refer to the murine monoclonal antibody developed by the inventors and characterized in Ghiotto M, Gauthier L, Serriari N, Pastor S, Truneh A, Nunes J A, et al. PD-L1 and PD-L2 differ in their molecular mechanisms of interaction with PD-1. International immunology. 2010; 22:651-60.

The 6 CDRs of the PD-L1.3.1 antibody are as in Table 1 below:

TABLE 1

| | DNA sequence | Aminoacid sequence |
|---|---|---|
| H-CDR1 | GACACCTATATGCAC | DTYMH |
| | (SEQ ID NO: 1) | (SEQ ID NO: 7) |
| H-CDR2 | TGGATTGATCCTGCGAATGGA | WIDPANGNTKYDPKFQG |
| | AATACCAAATATGACCCGAAG | (SEQ ID NO: 8) |
| | TTCCAGGGC | |
| | (SEQ ID NO: 2) | |
| H-CDR3 | TCTGGGGTTAGTACGGCCCAC | SGVSTAHFDY |
| | TTTGACTAC | (SEQ ID NO: 9) |
| | (SEQ ID NO: 3) | |
| L-CDR1 | AGGGCCAGCTCAAGTGTAAGT | RASSSVSFMH |
| | TTCATGCAC | (SEQ ID NO: 10) |
| | (SEQ ID NO: 4) | |
| L-CDR2 | GCCACATCCAACCTGGCTTCT | ATSNLAS |
| | (SEQ ID NO: 5) | (SEQ ID NO: 11) |
| L-CDR3 | CAGCAGTGGAGTAGTTACCCA | QQWSSYPRT |
| | CGGACG | (SEQ ID NO: 12) |
| | (SEQ ID NO: 6) | |

The complete sequences of the variable regions (VH and VL) of the PD-L1.3.1 mAb are the following:

Heavy chain: DNA sequence (414 bp): Leader
sequence-FR1-CDR1-FR2-CDR2-FR3-CDR3-FR4
(SEQ ID NO: 13)
ATGAAATGCAGCTGGGTTATCTTCTTCCTGATGGCAGTGGTTACAGGGGT
CAATTCAGAGGTTCAGCTGCAGCAGTCTGGGACAGAACTTGTGAAGCCAG
GGGCCTCAGTCAAGTTGTCCTGCACAACTTCTGGCTTCAACATTCAAGAC
ACCTATATGCACTGGGTGAAGCAGAGGCCTGAACAGGGCCTGGAGTGGAT
TGGATGGATTGATCCTGCGAATGGAAATACCAAATATGACCCGAAGTTCC
AGGGCAAGGCCACTATAATAGCAGACACATCCTCCAACACAGCCTACCTG
CAGCTCCGCGGCCTGACATCTGAGGACACTGCCGTCTATTACTGTGCTAG
ATCTGGGGTTAGTACGGCCCACTTTGACTACTGGGGCCAAGGCACCACTC
TCACAGTCTCCTCA
Heavy chain: Amino acids sequence (138 AA): Leader
sequence-FR1-CDR1-FR2-CDR2-FR3-CDR3-FR4
(SEQ ID NO: 14)
MKCSWVIFFLMAVVTGVNSEVQLQQSGTELVKPGASVKLSCTTSGFNIQD
TYMHWVKQRPEQGLEWIGWIDPANGNTKYDPKFQGKATIIADTSSNTAYL
QLRGLTSEDTAVYYCARSGVSTAHFDYWGQGTTLTVSS
Light chain: DNA sequence (384 bp): Leader
sequence-FR1-CDR1-FR2-CDR2-FR3-CDR3-FR4
(SEQ ID NO: 15)
ATGGATTTTCAAGTGCAGATTTTCAGCTTCCTGCTAATCAGTGCTTCAGT
CATAATGTCCAGAGGACAAATTGTTCTCTCCCAGTCTCCAGCAATCCTGT
CTGCATCTCCAGGGGAGAAGGTCACAATGACTTGCAGGGCCAGCTCAAGT
GTAAGTTTCATGCACTGGTACCAGCAGAAGCCAGGATCCTCCCCCAAACC
CTGGATTTATGCCACATCCAACCTGGCTTCTGGAGTCCCTACTCGCTTCA
GTGGCAGTGGGTCTGGGACCTCTTACTCTCTCACACTCAGCAGAGTGGAG
GCTGAAGATGCTGCCACTTATTACTGCCAGCAGTGGAGTAGTTACCCACG
GACGTTCGGTGGAGGCACCAAACTGGAAATCAAA
Light chain: Amino acids sequence (128 AA): Leader
sequence-FR1-CDR1-FR2-CDR2-FR3-CDR3-FR4
(SEQ ID NO: 16)
MDFQVQIFSFLLISASVIMSRGQIVLSQSPAILSASPGEKVTMTCRASSS
VSFMHWYQQKPGSSPKPWIYATSNLASGVPTRFSGSGSGTSYSLTLSRVE
AEDAATYYCQQWSSYPRTFGGGTKLEIK

The present invention also refers to antibodies comprising SEQ ID NO:18 in their heavy chain and SEQ ID NO:20 in their light chain. It also refers to antibodies encoded by at least nucleotidic sequences SEQ ID NO:17 for the heavy chain and SEQ ID NO:19 for the light chain:

Heavy chain: DNA sequence: FR1-CDR1-FR2-CDR2-FR3-
CDR3-FR4
(SEQ ID NO: 17)
GAGGTTCAGCTGCAGCAGTCTGGGACAGAACTTGTGAAGCCAGGGGCCTC
AGTCAAGTTGTCCTGCACAACTTCTGGCTTCAACATTCAAGACACCTATA
TGCACTGGGTGAAGCAGAGGCCTGAACAGGGCCTGGAGTGGATTGGATGG
ATTGATCCTGCGAATGGAAATACCAAATATGACCCGAAGTTCCAGGGCAA
GGCCACTATAATAGCAGACACATCCTCCAACACAGCCTACCTGCAGCTCC
GCGGCCTGACATCTGAGGACACTGCCGTCTATTACTGTGCTAGATCTGGG
GTTAGTACGGCCCACTTTGACTACTGGGGCCAAGGCACCACTCTCACAGT
CTCCTCA
Heavy chain: Amino acids sequence: FR1-CDR1-FR2-
CDR2-FR3-CDR3-FR4
(SEQ ID NO: 18)
EVQLQQSGTELVKPGASVKLSCTTSGFNIQDTYMHWVKQRPEQGLEWIGW
IDPANGNTKYDPKFQGKATIIADTSSNTAYLQLRGLTSEDTAVYYCARSG
VSTAHFDYWGQGTTLTVSS
Light chain: DNA sequence: FR1-CDR1-FR2-CDR2-FR3-
CDR3-FR4
(SEQ ID NO: 19)
CAAATTGTTCTCTCCCAGTCTCCAGCAATCCTGTCTGCATCTCCAGGGGA
GAAGGTCACAATGACTTGCAGGGCCAGCTCAAGTGTAAGTTTCATGCACT
GGTACCAGCAGAAGCCAGGATCCTCCCCCAAACCCTGGATTTATGCCACA
TCCAACCTGGCTTCTGGAGTCCCTACTCGCTTCAGTGGCAGTGGGTCTGG
GACCTCTTACTCTCTCACACTCAGCAGAGTGGAGGCTGAAGATGCTGCCA
CTTATTACTGCCAGCAGTGGAGTAGTTACCCACGGACGTTCGGTGGAGGC
ACCAAACTGGAAATCAAA
Light chain: Amino acids sequence: FR1-CDR1-FR2-
CDR2-FR3-CDR3-FR4
(SEQ ID NO: 20)
QIVLSQSPAILSASPGEKVTMTCRASSSVSFMHWYQQKPGSSPKPWIYAT
SNLASGVPTRFSGSGSGTSYSLTLSRVEAEDAATYYCQQWSSYPRTFGGG
TKLEIK

In one embodiment, the present invention relates to a labelled anti-PD-L1 antibody, having the following 6 CDRs:

| | Aminoacid sequence |
|---|---|
| H-CDR1 | DTYMH |
| | (SEQ ID NO: 7) |
| H-CDR2 | WIDPANGNTKYDPKFQG |
| | (SEQ ID NO: 8) |
| H-CDR3 | SGVSTAHFDY |
| | (SEQ ID NO: 9) |
| L-CDR1 | RASSSVSFMH |
| | (SEQ ID NO: 10) |
| L-CDR2 | ATSNLAS |
| | (SEQ ID NO: 11) |
| L-CDR3 | QQWSSYPRT |
| | (SEQ ID NO: 12) |

The anti-PD-L1 immunotoxin also comprises a toxin or a fragment thereof. In one embodiment, said toxin or its fragment is a Ribosome Inactivating Protein (RIP).

Typically, the Ribosome Inactivating Protein is chosen from saporin, ricin, abrin, gelonin, Pseudomonas exotoxin (or exotoxin A), trichosanthin, luffin, agglutinin and the diphtheria toxin. In another embodiment, the toxin may also be a chemical drug.

Typically, the toxin is chosen from modeccin, mitogellin, chlortetracycline, mertansine, monomethyl auristatin E, monomethyl auristatin F, and enediynes, especially calicheamicins (like calicheamicin k or calicheamicin γl) and their related esperamicins (like esperamicin A1). Enediynes are chemical compounds characterized by either 9- or 10-membered rings containing two triple bonds separated by a double bond.

In a particular embodiment, the toxin is selected in the group consisting of mertansine (DM1), monomethyl auristatin E (MMAE) and monomethyl auristatin F (MMAF).

When the toxin is mertansine, it is linked to the antibody or a fragment thereof by a linker. When the linker is 4-mercaptovaleric acid, the group comprising the toxin and the linker is called emtansine. Mertansine refers to the thiol-containing maytansinoid, DM1 (N2′-deacetyl-N2′-(3-mercapto-1-oxopropyl)maytansine) attached to a monoclonal antibody through reaction of the thiol group with the SPP (N-succinimidyl 4-(2-pyridyldithio)) linker to create an antibody-drug conjugate or ADC. In a preferred embodiment, the toxin is DM1.

When the toxin is monomethyl auristatin E (MMAE), it is linked to the antibody or a fragment thereof by a structure comprising a spacer (which is preferably paraaminobenzoic acid), a cathepsin-cleavable linker (preferably consisting of citrulline and valine) and an attachment group or linker (preferably consisting of 6-maleimidocaproic acid). Preferably in such a case, the group comprising the toxin and the structure as defined in the previous sentence is vedotin.

When the toxin is monomethyl auristatin F (MMAF), it is linked to the antibody or a fragment thereof by a structure comprising an attachment group or linker (preferably consisting of 6-maleimidocaproic acid). Preferably in such a case, the group comprising the toxin and the structure as defined in the previous sentence is mafodotin.

The toxin may also be chosen from anticancer agents. Said anticancer agents can be chosen from combrestatin, colchicine, actinomycine, duocarmycins and their synthetic analogues (adozelesin, bizelesin and carzelesin), fludarabine, gemcitabine, capecitabine, methotrexate, taxol, taxotere, mercaptopurine, thioguanine, hydroxyurea, cytarabine, cyclophosphamide, ifosfamide, platinum complexes (such as cisplatin, carboplatin and oxaliplatin), mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epimbicin, 5-fluorouracil, taxanes (such as docetaxel and paclitaxel), leucovorin, levamisole, irinotecan, estramustine, etoposide, nitrogen mustards, BCNU, nitrosoureas (such as carmustine and lomustine), vinca alkaloids (such as vinblastine, vincristine, dolastatins and vinorelbine), imatinib mesylate, hexamethylenediamine, topotecan, kinase inhibitors (like the tyrosine kinase inhibitors called tyrphostins), phosphatase inhibitors, ATPase inhibitors, protease inhibitors, inhibitors of herbimycin A, genistein, erbstatin, and lavendustin A.

The toxin may also be a radioisotope, preferably chosen from ²¹¹At, ¹³¹I, ¹²⁵I, ¹⁸⁶Re, ¹⁸⁸Re, ¹⁵³Sm, P³², ⁹⁰Y, ¹⁷⁷Lu, ⁶⁷Cu, ⁴⁷Sc, ²¹²Bi, ²¹³Bi, ²²⁶Th, ¹¹¹In and ⁶⁷Ga.

In one embodiment, the immunotoxin is the anti-PD-L1 antibody PD-L1.3.1 linked to auristatin or DM1.

As used herein, the term “tumor” refers to an abnormal mass or population of cells that result from excessive cell division, whether malignant or benign, and all pre-cancerous and cancerous cells and tissues. A tumor can be solid tumor or a haematological malignancy.

Examples of solid tumors include, but are not limited, to prostate cancer, pancreatic cancer, breast cancer, melanoma, B cell lymphoma, brain cancer, bladder cancer, colon cancer, intestinal cancer, lung cancer, stomach cancer, cervical cancer, ovarian cancer, liver cancer, skin cancer, colorectal cancer, endometrial carcinoma, salivary gland carcinoma, kidney cancer, thyroid cancer, various types of head and neck cancers.

In one embodiment of the invention, said solid tumor is selected among prostate, breast and pancreatic cancers.

In another embodiment, the tumor is a hematological malignancy selected from B-cell lymphoid neoplasm, T-cell lymphoid neoplasm, non-Hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm and myeloid cell lineage neoplasm.

As used herein, the term “subject” denotes a mammal, such as a rodent, a feline, a canine, and a primate. Preferably a subject according to the invention is a human.

According to the invention, the term “patient” or “patient in need thereof” is intended for a human or non-human mammal affected or likely to be affected by a tumor.

As used herein, the term “PD-L1-positive tumor” denotes a tumor which expresses PD-L1 and is therefore likely to respond to a therapy which targets PD-L1 or its ligand PD-1.

Methods for determining whether a solid tumor is a PD-L1-positive tumor have described in the art. Typically, a tumor sample (e.g. a biopsy sample) obtained from a patient can be contacted with a PD-L1 ligand, such as a PD-L1 antibody.

Suitable methods are described for instance in WO 2010/089411.

In one aspect, the present invention relates to a method for treating a patient suffering from a tumor, preferably a PD-L1-postive tumor, comprising the step of administering to said patient a therapeutically effective amount of anti-PD-L1 immunotoxin as described above.

By a “therapeutically effective amount” of the anti-PD-L1 immunotoxin according to the invention is meant a sufficient amount of said immunotoxin to treat said solid tumor, at a reasonable benefit/risk ratio applicable to any medical treatment. It will be understood, however, that the total daily usage of immunotoxin and compositions of the present invention will be decided by the attending physician within the scope of sound medical judgment. The specific therapeutically effective dose level for any particular patient will depend upon a variety of factors including the disorder being treated and the severity of the disorder, activity of the specific antagonist of the active employed; the specific composition employed, the age, body weight, general health, sex and diet of the patient, the time of administration, route of administration, and rate of excretion of the specific antibody employed, the duration of the treatment; drugs used in combination or coincidental with the specific polypeptide employed, and like factors well known in the medical arts. For example, it is well known within the skill of the art to start doses of the compound at levels lower than those required to achieve the desired therapeutic effect and to gradually increase the dosage until the desired effect is achieved.

A further object of the invention relates to a pharmaceutical composition comprising an anti-PD-L1 immunotoxin.

Any therapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of antagonist of the actives i) and ii) may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaC1 solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g. tablets or other solids for oral administration; time release capsules; and any other form currently used.

Compositions of the present invention may comprise a further therapeutic active agent. The present invention also relates to a kit comprising an anti-PD-L1 immunotoxin as defined above and a further therapeutic active agent.

In one embodiment said therapeutic active agent is an anticancer agent. For example, said anticancer agents include but are not limited to fludarabine, gemcitabine, capecitabine, methotrexate, taxol, taxotere, mercaptopurine, thioguanine, hydroxyurea, cytarabine, cyclophosphamide, ifosfamide, platinum complexes such as cisplatin, carboplatin and oxaliplatin, mitomycin, dacarbazine, procarbizine, etoposide, teniposide, campathecins, bleomycin, idarubicin, daunorubicin, dactinomycin, plicamycin, mitoxantrone, L-asparaginase, doxorubicin, epimbicin, 5-fluorouracil, taxanes such as docetaxel and paclitaxel, leucovorin, levamiso le, irinotecan, estramustine, etoposide, nitrogen mustards, BCNU, nitrosoureas such as carmustine and lomustine, vinca alkaloids such as vinblastine, vincristine and vinorelbine, imatinib mesylate, hexamethylenediamine, topotecan, kinase inhibitors, phosphatase inhibitors, ATPase inhibitors, tyrphostins, protease inhibitors, inhibitors of herbimycin A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, anthracyclines, MDR inhibitors and Ca2+ ATPase inhibitors.

Additional anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or muti-specific antibodies, monobodies, polybodies.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzoquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinols, thiethylperazine, thioproperazine and tropisetron. In a preferred embodiment, the antiemetic agent is granisetron or ondansetron.

In still another embodiment, the other therapeutic active agent can be an opioid or non-opioid analgesic agent Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflusinal, etodolac, fenoprofen, flurbiprofen, ibuprofen, ketoprofen, indomethacin, ketorolac, meclo fenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In one aspect, the present invention relates to a kit-of-parts comprising i) an anti-PD-L1 immunotoxin, and ii) an anti-cancer agent, as a combined preparation for simultaneous, separate or sequential use in the treatment of a tumor.

The invention will be further illustrated through the following examples and figures.

### FIGURES LEGENDS

FIG. 1. A: Binding of ¹¹¹In-PD-L1.3.1 to five different breast cancer cell lines. B: IC₅₀ analysis of PD-L1.3.1. C: Scatchard analysis of ¹¹¹In-PD-L1.3.1. D: Internalization kinetics of ¹¹¹In-PD-L1.3.1.

FIG. 2. A: Dose escalation study of ¹¹¹In-PD-L1.3.1 in mice with subcutaneous MDA-MB-231 xenografts, 3 days p.i. B: Tumor uptake of ¹¹¹In-PD-L1.3.1 (1 μg) in mice with subcutaneous MDA-MB-231 or MCF-7 xenografts. Separate groups of mice were injected with an excess of unlabeled PD-L1.3.1.

FIG. 3. A: Typical examples of SPECT/CT scans of mice with subcutaneous MDA-MB-231 or MCF-7 xenografts, acquired at different time-points after injection of 15.5 MBq ¹¹¹In-PD-L1.3.1 (1.5 μg). Tumors are indicated with the white arrows. B: Close-up of the heterogeneous targeting of ¹¹¹In-PD-L1.3.1 in MDA-MB-231 xenografts. C: Uptake of ¹¹¹In-PD-L1.3.1 in the tumor and liver of mice bearing subcutaneous MDA-MB-231 or MCF-7 xenografts, as quantified from the SPECT scans.

FIG. 4. SPECT/CT scans of mice bearing subcutaneous breast cancer xenografts, with different PD-L1 expression levels, on both flanks (indicated with the white arrows). Scans were acquired three days post injection of 10 MBq ¹¹¹In-PD-L1.3.1 (1 μg).

FIG. 5. Examples of autoradiography and cross section of SPECT scans to illustrate the heterogeneous distribution of ¹¹¹In-PD-L1.3.1 in the xenograft, HE staining, and PD-L1 immunostaining of breast cancer xenografts.

### EXAMPLES

### Example 1

### Coupled Anti-PD-L1 Antibodies are Effectively Targeted to PD-L1-Positive Tumors.

Material and Methods

Cell Culture

The breast cancer cell lines MDA-MB-231, SK-Br-3, and MCF-7 were cultured in RPMI1640 (GIBCO, BRL Life Sciences Technologies, The Netherlands), supplemented with 2 mM glutamine (GIBCO) and 10% FCS (Sigma-Aldrich Chemie BV, The Netherlands) at 37° C. in a humidified atmosphere with 5% CO₂. SUM149 was cultured in Ham's F12 medium (GIBCO) supplemented with 5% FCS, 10 mM 4-(2-hydroxyethyl)-1-piperazineethanesulfonic acid (HEPES, GIBCO), hydrocortisone (1 μg/ml, Sigma-Aldrich Chemie BV), and insulin (5 μg/ml, Sigma-Aldrich Chemie BV). BT474 was cultured in RPMI1640, 2 mM glutamine, 10% FCS, and 10 μg/ml insulin.

FACS Analysis of PD-L1 Expression

PD-L1 expression of MDA-MB-231, SK-Br-3, SUM149, BT474, and MCF-7 cells was determined by FACS analysis. Cells were incubated with PD-L1-PE (557924, BD biosciences, San Jose, Calif.) or mouse IgG1-PE (400114, Biolegend, San Diego, Calif.) for 30 min at 4° C. Cells were washed and, subsequently, analyzed using the Gallios flow cytometer (Beckman Coulter, Fullerton, Calif., USA).

Radiolabeling

The murine monoclonal IgG1 antibody PD-L1.3.1 is specifically directed against human PD-L1 and does not cross react with murine PD-L1.(21) It was conjugated with isothiocyanatobenzyl-diethylenetriaminepentaacetic acid (ITC-DTPA, Macrocyclis, Dallas, Tex.) in 0.1 M NaHCO₃, pH 9.5, at a 21-fold molar excess of ITC-DTPA, for 1 h at room temperature (RT). Unbound ITC-DTPA was removed from the reaction mixture by dialysis against 0.1 M 2-(N-morpholino)ethanesulfonic acid (MES, Sigma-Aldrich Chemie BV) buffer, followed by purification on a disposable G25M Sephadex column (PD10, GE Healthcare Life Sciences, Eindhoven, The Netherlands), eluted with 0.25 M ammoniumacetate buffer, pH 5.4 (Sigma-Aldrich Chemie BV).

DTPA-conjugated PD-L1.3.1 was incubated with ¹¹¹In (Mallinckrodt BV, Petten, The Netherlands) in 0.5 M MES buffer, pH 5.4, 20 min at RT, under strict metal-free conditions.(22) After incubation, 50 mM ethylenediaminetetraacetic acid (EDTA) was added to a final concentration of 5 mM to chelate unincorporated ¹¹¹In.

Labeling efficiency was determined using instant thin-layer chromatography (ITLC) on silica gel chromatography strips (Agilent Technologies, Palo Alto, Calif.), using 0.1 M citrate buffer (Sigma-Aldrich Chemie BV), pH 6.0, as the mobile phase. In case labeling efficiency was below 95%, the reaction mixture was purified on a PD-10 column, eluted with PBS, containing 0.5% BSA (Sigma-Aldrich Chemie BV). Radiochemical purity of ¹¹¹In-DTPA-PD-L1.3.1 (¹¹¹In-PD-L1.3.1) exceeded 95% in all experiments.

In Vitro Assays

Binding to Breast Cancer Cell Lines

The breast cancer cell lines MDA-MB-231, SK-Br-3, SUM149, BT474, and MCF-7 were cultured to confluency in six-well plates and incubated with 32 pM ¹¹¹In-PD-L1.3.1 (1 kBq) for 4 h at 37° C. in a humidified atmosphere with 5% CO₂, or for 4 h on ice, in RPMI1640 containing 0.5% BSA. Separate wells were coincubated with a 1,000-fold excess of unlabeled PD-L1.3.1 to determine non-specific binding. After incubation, cells were washed with PBS and the cell-associated activity was measured in a shielded well-type gamma counter (Perkin-Elmer, Boston, Mass., USA). Specific binding was calculated by subtracting the non-specific binding from the total binding.

Immunoreactive Fraction

The immunoreactive fraction (IRF) of ¹¹¹In-PD-L1.3.1 was determined essentially as described by Lindmo et al (26). A serial dilution of MDA-MB-231 cells (3.3×10⁵-8.4−10⁷ cells/ml) in RPMI1640 containing 0.5% BSA was incubated with 8 pM ¹¹¹In-PD-L1.3.1 (0.2 kBq). Non-specific binding was determined by adding an excess of unlabeled PD-L1.3.1 (67 nM) to a duplicate of the lowest cell concentration. After 1 h incubation at 37° C., cells were centrifuged and the activity in the cell pellet was measured in a shielded 3-inch-well-type gamma counter (Perkin-Elmer, Boston, Mass., USA). The inverse of the specific cell bound activity was plotted against the inverse of the cell concentration, and the immunoreactive fraction was calculated from the y-axis intercept using GraphPad Prism (version 5.03 for Windows).

IC₅₀

MDA-MB-231 cells were cultured to confluency in six-well plates. The 50% inhibitory concentration (IC₅₀) of PD-L1.3.1 blocking of ¹¹¹In-PD-L1.3.1 binding was determined by incubating the cells for 4 h on ice in 1 ml RPMI1640 0.5% BSA, containing 20 pM ¹¹¹In-PD-L1.3.1 (1 kBq) and increasing concentrations of unlabeled PD-L1.3.1 (1-1000 pM). After incubation, cells were washed with PBS and the cell-associated activity was measured in a gamma counter. The IC₅₀ was defined as the antibody concentration that was required to inhibit binding of the radio labeled antibody by 50%. IC₅₀ values were calculated using GraphPad Prism.

Scatchard Analysis

Scatchard analysis was performed to determine the dissociation constant (K_{d}) of ¹¹¹In-PD-L1.3.1 and to quantitatively measure PD-L1 expression on MDA-MB-231, SK-Br-3, and SUM149 cells. Cells were cultured to confluency in six-well plates and were incubated for 4 h on ice with increasing concentrations ¹¹¹In-PD-L1.3.1 (3-3000 pM) in 1 ml RPMI1640 containing 0.5% BSA. Non-specific binding was determined by co-incubation with 100 nM PD-L1.3.1. After incubation, cells were washed with PBS and the cell-associated activity was measured in a shielded well-type gamma counter. The specific binding (total binding−nonspecific binding) was plotted against the bound/free ratio. Data were analyzed by linear regression to determine PD-L1 receptor density per cell and to determine the K_{d} of ¹¹¹In-PD-L1.3.1.

Internalization Kinetics

MDA-MB-231 cells were cultured in six-well plates and were incubated for 2, 4, or 24 h with 75 pM ¹¹¹In-PD-L1.3.1 (1 kBq) in RPMI1640 containing 0.5% BSA at 37° C. in a humidified atmosphere with 5% CO₂. Nonspecific binding and internalization was determined by coincubation with 17 nM unlabeled PD-L1.3.1. After incubation, acid wash buffer (0.1 M HAc, 0.15 M NaCl, pH 2.6) was added for 10 min to remove the membrane-bound fraction of the cell-associated ¹¹¹In-PD-L1.3.1. Subsequently, cells were harvested from the six-well plates and the amount of membrane bound and internalized activity was measured in a gamma counter. Specific binding and internalization were calculated by subtracting the non-specific binding and internalization from the total binding and internalization.

Animal Studies

Animal experiments were performed on female BALB/c nude mice (Janvier, le Genest-Saint-Isle, France) and were conducted in accordance with the principles laid out by the revised Dutch Act on Animal Experimentation (1997) and approved by the institutional Animal Welfare Committee of the Radboud University Nijmegen. At 6-8 weeks of age, mice were inoculated subcutaneously with 5×10⁶ MDA-MB-231, SK-Br-3, SUM149, MCF-7, or BT474 cells (mixed 2:1 with matrigel, BD Biosciences, Pharmingen). Mice receiving MCF-7 or BT474 cells were, prior to tumor cell inoculation, implanted subcutaneously with a slow release estradiol pellet (0.18 mg, 60 days, Innovative Research of America, Sarasote, Fla.) under general anesthesia (isoflurane/O₂). Experiments started when tumors reached a size of approximately 0.1 cm³.

Dose Optimization

Seven groups (n=6) of mice with subcutaneous MDA-MB-231 xenografts received an intravenous injection of 0.2 MBq ¹¹¹In-PD-L1.3.1 (specific activity 0.4 MBq/μg) in the tail vein. To study the effect of the antibody protein dose on the biodistribution of ¹¹¹In-PD-L1.3.1, groups received increasing protein doses of PD-L1.3.1 (0.3-300 μg/mouse). Three days post injection, mice were euthanized using CO₂/O₂-asphyxiation. The biodistribution of the radiolabel was determined ex vivo. Tumor, blood, muscle, lung, heart, spleen, pancreas, intestine, kidney, liver, bone, and bone marrow were dissected and weighed. Activity was measured in a gamma counter. To determine the uptake of radiolabeled antibodies in each sample as a fraction of the injected dose, aliquots of the injected dose were counted simultaneously. The results were expressed as percentage injected dose per gram tissue (% ID/g).

Biodistribution Studies

Three groups (n=6) of mice with subcutaneous MDA-MB-231 xenografts and three groups with MCF-7 xenografts received an intravenous injection of 0.2 MBq ¹¹¹In-PD-L1.3.1. Separate groups of mice were coinjected with an excess of 300 μg unlabeled PD-L1.3.1 to block PD-L1 in vivo. At 1, 3 and 7 days post injection of radiolabeled PD-L1.3.1, mice were euthanized and the ex vivo biodistribution of radiolabeled PD-L1.3.1 was determined as described previously.

SPECT/CT Imaging

Three mice with subcutaneous MDA-MB-231 and three mice with subcutaneous MCF-7 xenografts received an intravenous injection of 15.5 MBq ¹¹¹In-PD-L1.3.1 (protein dose 1.5 μg). Immediately after injection and 1, 3 and 7 days post injection, images were acquired with the U-SPECT-II/CT (MILabs, Utrecht, The Netherlands).(23) Mice were scanned under general anesthesia (isoflurane/O₂) for 30-90 min using the 1.0 mm diameter pinhole mouse high sensitivity collimator tube, followed by a CT scan (spatial resolution 160 μm, 65 kV, 615 μA) for anatomical reference. Scans were reconstructed with MILabs reconstruction software, using an ordered-subset expectation maximization algorithm, with a voxel size of 0.2 mm. SPECT/CT scans were analyzed and maximum intensity projections (MIPs) were created using the Inveon Research Workplace software (IRW, version 4.1). A 3D volume of interest was drawn around the tumor and uptake was quantified as the percentage injected dose per gram (% ID/g), assuming a tissue density of 1 g/cm³.

¹¹¹In-PD-L1 SPECT/CT in Breast Cancer Models with Different PD-L1Expression Levels

Tumor targeting of ¹¹¹In-PD-L1.3.1 to MDA-MB-231, SK-Br-3, SUM149, MCF-7, and BT474 xenografts was determined in 10 mice bearing subcutaneous tumors on both flanks (n=2 mice/4 tumors per xenograft model). Mice received intravenous tail vein injections of 1 μg (10 MBq) ¹¹¹In-PD-L1.3.1 and 3 days later, mice were euthanized by CO₂/O₂ asphyxation and SPECT/CT images were acquired for 90 min, as described previously. Subsequently, the ex vivo biodistribution of radiolabeled PD-L1.3.1 was determined. Tumors were fixed in 4% formalin or frozen at −80° C. for autoradiography and immunohistochemistry.

Autoradiography

Frozen and formalin-fixed tumor sections (5 gm) from mice injected with 10 MBq of ¹¹¹In-PD-L1.3.1 (1 μg) were exposed to a Fujifilm BAS cassette 2025 overnight (Fuji Photo Film). Phospholuminescence plates were scanned using a Fuji BAS-1800 II bioimaging analyzer at a pixel size of 50×50 μm. Images were analyzed with Aida Image Analyzer software (Raytest).

Immunohistochemistry

Frozen tumor sections of MDA-MB-231, SK-Br-3, SUM149, BT474, and MCF-7 were fixed for 10 min in ice cold acetone (−20° C.). Endogenous mouse Ig staining was blocked using a mouse-on-mouse blocking kit (BMK-2202, Vector). Subsequently, sections were incubated with 10 μg/ml PD-L.3.1, followed by incubation with a peroxidase-conjugated rabbit-anti-mouse antibody (P0260, DAKO). Finally, 3-3′-Diaminobenzidine (DAB) was used to visualize peroxidase activity in the sections.

Statistical Analyses

Statistical analyses were performed using PASW Statistics version 18.0 (Chicago, Ill.) and GraphPad Prism version 5.03 (San Diego, Calif.) for Windows. Differences in uptake of radiolabeled PD-L1.3.1 were tested for significance using the nonparametric Kruskal-Wallis and Mann-Whitney U test. The correlation between tumor uptake measured by SPECT and ex vivo biodistribution was calculated with the Spearman correlation coefficient. A p-value below 0.05 was considered significant.

Results

¹¹¹In-PD-L1.3.1 Specifically Binds to PD-L1

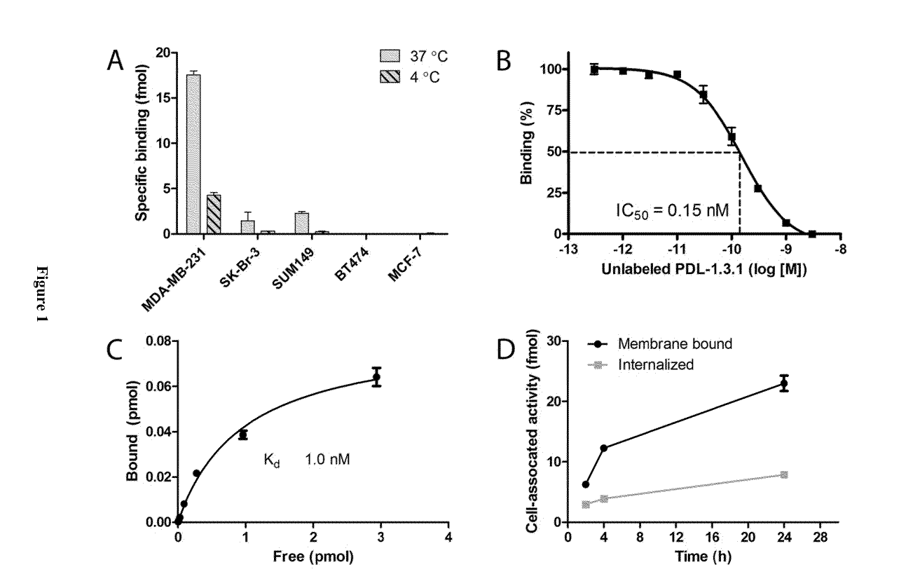

Flow cytometry analysis showed that the percentage of tumor cells positive for PD-L1 was 89.4%, 2.9%, 8.9%, 0.2%, and 0.1% for MDA-MB-231, SK-Br-3, SUM149, BT474, and MCF-7, respectively. PD-L1.3.1 was labeled with ¹¹¹In, obtaining specific activities up to 10 MBq/μg antibody. ¹¹¹In-PD-L1.3.1 showed the highest binding to MDA-MB-231 cells, while binding to SUM149 and SK-Br-3 was significantly lower (P=0.002). PD-L1 negative BT474 and MCF-7 cells did not show any specific binding of ¹¹¹In-PD-L1.3.1 (FIG. 1A). The number of binding sites for PD-L1.3.1 was determined quantitatively with scatchard analysis and was 47,700±2,900 for MDA-MB-231, 2,000±100 for SK-Br-3, and 3,600±400 for SUM149. Based on these results MDA-MB-231 cells were used in subsequent binding assays.

The IRF of ¹¹¹In-PD-L1.3.1 was 82% and the IC₅₀ of unlabeled PD-L1.3.1 was 0.15 nM (FIG. 1B). Scatchard analysis showed that the affinity of ¹¹¹In-labeled PD-L1.3.1 was 0.97±0.15 nM (FIG. 1C). PD-L1.3.1 was slowly internalized by MDA-MB-231 cells. After 24 h of incubation, 25% of the cell-associated activity was internalized and 75% was still membrane-bound (FIG. 1D). These data demonstrate that ¹¹¹In-PD-L1.3.1 antibody specifically binds to PD-L1 expressing tumor cells.

¹¹¹In-PD-L1.3.1 Accumulates Specifically in PD-L1 Positive Xenografts

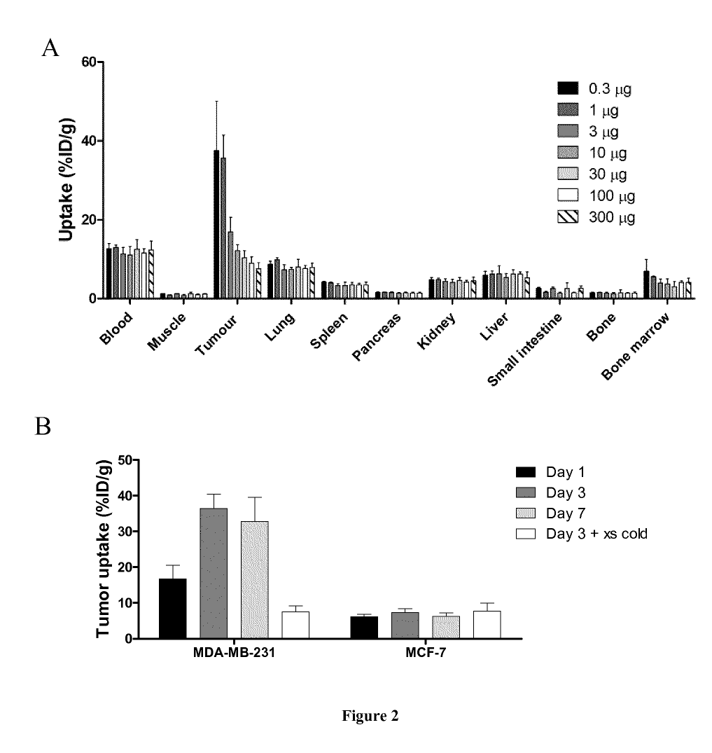

The antibody protein dose escalation study showed high and specific tumor accumulation of ¹¹¹In-PD-L1.3 (FIG. 2A). Tumor uptake in PD-L1 positive MDA-MB-231 was the highest in mice injected with 0.3 or 1 μg of antibody (37.5±12.5 and 35.7 ±5.8% ID/g, respectively, FIG. 2A). At antibody doses of ≧3 μg, tumor uptake significantly decreased (3 μg: 16.9±3.8% ID/g, p=0.002). Tumor uptake was the lowest in mice injected with 300 μg PD-L1.3.1 (7.6±1.6% ID/g). Other organs did not show specific targeting of ¹¹¹In-PD-L1.3.1.

Uptake of ¹¹¹In-PD-L1.3.1 by PD-L1 positive MDA-MB-231 xenografts was observed as early as 1 day post injection and further increased at day 3 and 7 (Table 2, FIG. 2B). PD-L1 negative MCF-7 xenografts did not show specific uptake at any time point. Tumor-to-blood ratios increased over time for MDA-MB-231 and were the highest 7 days post injection (3.9±1.0). Tumor-to-blood ratios for MCF-7 did not exceed 0.9 and were not significantly increased compared with the tumor-blood-ratios in mice that received an excess of unlabeled PD-L1.3.1. These data demonstrate that ¹¹¹In-PD-L1.3.1 can discriminate between PD-L1 positive and PD-L1 negative xenografts.

TABLE 2
Tumor targeting of ¹¹¹In-PD-L1.3.1 to MDA-MB-231 and MCF-7
xenografts at 1, 3, and 7 days post injection.

| | Tumor | Tumor-to |
|---|---|---|
| Xenograft | uptake (% ID/g) | blood ratio |
| MDA-MB-231 | | |
| Day 1 | 16.8 ± 3.8 | 1.1 ± 0.2 |
| Day 3 | 36.4 ± 4.0 | 3.1 ± 0.2 |
| Day 7 | 32.8 ± 6.8 | 3.9 ± 1.0 |
| Day 3 + excess unlabeled | 7.6 ± 1.6 | 0.6 ± 0.1 |
| MCF-7 | | |
| Day 1 | 6.1 ± 0.7 | 0.5 ± 0.1 |
| Day 3 | 7.3 ± 1.0 | 0.8 ± 0.1 |
| Day 7 | 6.2 ± 1.0 | 0.9 ± 0.2 |
| Day 3 + excess unlabeled | 7.7 ± 2.2 | 0.8 ± 0.2 |

SPECT/CT Visualizes PD-L1 Positive Xenografts

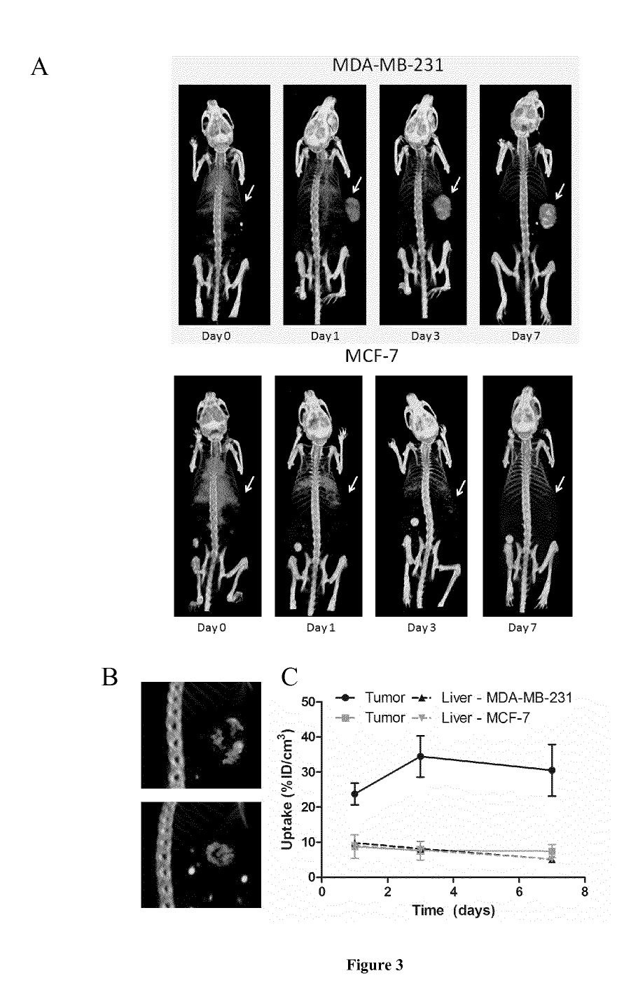

MDA-MB-231 xenografts were clearly visualized with ¹¹¹In-PD-L1.3.1 SPECT/CT, with increasing contrast between the tumor and normal tissue with time. Typical examples of SPECT/CT scans, and the quantification of tumor and liver uptake are presented in FIG. 3. Tumor uptake in MDA-MB-231 xenografts increased over time, while the uptake in MCF-7 xenografts did not exceed uptake in normal organs, such as the liver. Intratumoral distribution of ¹¹¹In-PD-L1.3.1 was heterogeneous, as is visualized in a zoomed high-resolution image of the tumor in FIG. 3B. There was a strong correlation between the tumor uptake as measured by SPECT and by counting of dissected tissues at 7 days p.i. (Spearman r=0.94).

¹¹¹In-PD-L1 SPECT/CT Can Discriminate Xenografts with High and Low PD-L1 Expression Levels

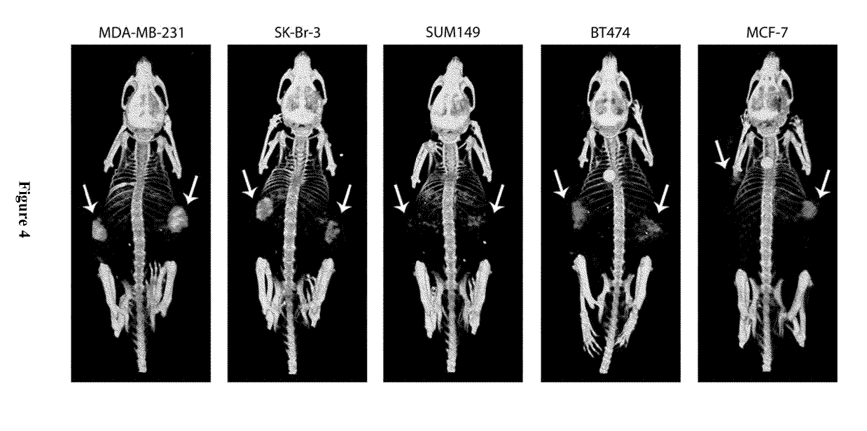

SPECT/CT images demonstrated high uptake of ¹¹¹In-PD-L1.3.1 in MDA-MB-231 and SK-Br-3 xenografts and low uptake in SUM149, BT474, and MCF-7 xenografts (FIG. 4), which was confirmed in the ex vivo biodistribution study. Tumor uptake at 3 days post injection was 25.2±2.9% ID/g, 22.0±5.1% ID/g, 8.4±0.2% ID/g, 10.0±0.7% ID/g, and 8.1±1.4% ID/g, for MDA-MB-231, SK-Br-3, SUM149, BT474, and MCF-7 xenografts, respectively.

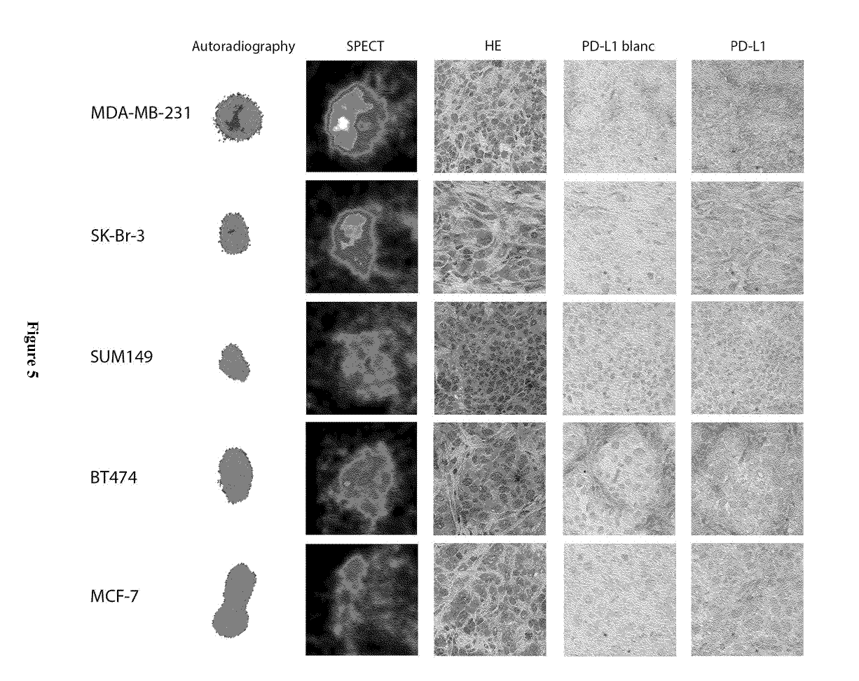

Autoradiographical analysis of the tumor sections showed that ¹¹¹In-PD-L1.3.1 antibody was distributed heterogeneously within the tumor. In general, highest tumor uptake was observed in the periphery of the tumor, while the uptake in the tumor center was lower. This heterogeneous distribution was also observed on cross-sections of the SPECT/CT scan (FIG. 5). After autoradiographical analysis, the same slides were used for HE staining which showed that the tumors contained areas with vital tissue (mostly in the periphery of the tumor) and areas with necrosis (mostly in the center of the tumor). In MDA-MB-231 and SK-Br-3 xenografts, highest uptake of ¹¹¹In-PD-L1.3.1 was found in the vital part of the tumor.

Immunohistochemical analysis of tumor sections for PD-L1 expression showed that MDA-MB-231 tumors expressed the highest levels of PD-L1. SK-Br-3 xenografts also clearly expressed PD-L1, although the expression levels varied largely between the tumors. Immunostaining of SUM149, BT474, and MCF-7 showed low PD-L1 expression (FIG. 5).

In summary, ¹¹¹In-PD-L1.3.1 SPECT/CT can discriminate between xenografts with high and low PD-L1 expression levels.

Conclusion

The inventors have shown that the labelled antibodies of the present invention are able to target PD-L1-positive tumors. They can be conjugated to a toxin (e.g. ¹¹¹In) without losing their functional properties.

### Example 2

### Production of Anti-PD-L1 Immunotoxins

Immunotoxin conjugated mAb is obtained by conjugating the PD-L1.3.1 antibody to DM1 or Auristatin. The antibody-drug conjugate (ADC) obtained is tested on cells plated in 96 well plate at 20-30% confluency in 100 μl culture medium and incubated overnight.

Serial dilution of antibody are done from 20 nM to 160 nM and added to cells. Protein G-drug conjugate is then added (20 nM) to appropriate wells. Plates are incubated 4 days and cell viability measured using the alamar bue assay. Significant cytotoxicity is observed on several tumor cell lines, such as the prostate cancer cell line PC3.

### REFERENCES

Throughout this application, various references describe the state of the art to which this invention pertains. The disclosures of these references are hereby incorporated by reference into the present disclosure.

1. Riella L V, Paterson A M, Sharpe A H, Chandraker A. Role of the PD-1 pathway in the immune response. American journal of transplantation: official journal of the American Society of Transplantation and the American Society of Transplant Surgeons. 2012; 12:2575-87.

2. Hamid O, Carvajal R D. Anti-programmed death-1 and anti-programmed death-ligand 1 antibodies in cancer therapy. Expert Opin Biol Ther. 2013; 13:847-61.

3. Saresella M, Rainone V, Al-Daghri N M, Clerici M, Trabattoni D. The PD-1/PD-L1 pathway in human pathology. Current molecular medicine. 2012; 12:259-67.

4. Ostrand-Rosenberg S, Horn L A, Haile S T. The Programmed Death-1 Immune-Suppressive Pathway: Barrier to Antitumor Immunity. J Immunol. 2014; 193:3835-41.

5. Zou W, Chen L. Inhibitory B7-family molecules in the tumour microenvironment. Nature reviews Immunology. 2008; 8:467-77.

6. Brahmer J R, Tykodi S S, Chow L Q, Hwu W J, Topalian S L, Hwu P, et al. Safety and activity of anti-PD-L1 antibody in patients with advanced cancer. N Engl J Med. 2012; 366:2455-65.

7. Topalian S L, Hodi F S, Brahmer J R, Gettinger S N, Smith D C, McDermott D F, et al. Safety, activity, and immune correlates of anti-PD-1 antibody in cancer. N Engl J Med. 2012; 366:2443-54.

8. Lipson E J, Sharfman W H, Drake C G, Wollner I, Taube J M, Anders R A, et al. Durable cancer regression off-treatment and effective reinduction therapy with an anti-PD-1 antibody. Clin Cancer Res. 2013; 19:462-8.

9. Hamid O, Robert C, Daud A, Hodi F S, Hwu W J, Kefford R, et al. Safety and tumor responses with lambrolizumab (anti-PD-1) in melanoma. N Engl J Med. 2013; 369:134-44.

10. Topalian S L, Sznol M, McDermott D F, Kluger H M, Carvajal R D, Sharfman W H, et al. Survival, durable tumor remission, and long-term safety in patients with advanced melanoma receiving nivolumab. J Clin Oncol. 2014; 32:1020-30.

11. Robert C, Ribas A, Wolchok J D, Hodi F S, Hamid O, Kefford R, et al. Anti-programmed-death-receptor-1 treatment with pembrolizumab in ipilimumab-refractory advanced melanoma: a randomised dose-comparison cohort of a phase 1 trial. Lancet. 2014.

12. Taube J M, Klein A, Brahmer J R, Xu H, Pan X, Kim JH, et al. Association of PD-1, PD-1 Ligands, and Other Features of the Tumor Immune Microenvironment with Response to Anti-PD-1 Therapy. Clin Cancer Res. 2014.

13. Grosso J, Horak C E, Inzunza D, Cardona D M, Simon J S, Gupta A K, et al. Association of tumor PD-L1 expression and immune biomarkers with clinical activity in patients (pts) with advanced solid tumors treated with nivolumab (anti-PD-1; BMS-936558; ONO-4538). Journal of Clinical Oncology. 2013; 31.

14. Herbst R S, Gordon M S, Fine G D, Sosman J A, Soria J C, Hamid O, et al. A study of MPDL3280A, an engineered PD-L1 antibody in patients with locally advanced or metastatic tumors. Journal of Clinical Oncology. 2013; 31.

15. Dong H, Strome S E, Salomao D R, Tamura H, Hirano F, Flies D B, et al. Tumor-associated B7-H1 promotes T-cell apoptosis: a potential mechanism of immune evasion. Nature medicine. 2002; 8:793-800.

16. Joseph R W, Parasramka M, Eckel-Passow J E, Serie D, Wu K, Jiang L, et al. Inverse association between programmed death ligand 1 and genes in the VEGF pathway in primary clear cell renal cell carcinoma. Cancer immunology research. 2013; 1:378-85.

17. Kondo A, Yamashita T, Tamura H, Zhao W, Tsuji T, Shimizu M, et al. Interferon-gamma and tumor necrosis factor-alpha induce an immunoinhibitory molecule, B7-H1, via nuclear factor-kappaB activation in blasts in myelodysplastic syndromes. Blood. 2010; 116:1124-31.

18. Ghebeh H, Lehe C, Barhoush E, Al-Romaih K, Tulbah A, Al-Alwan M, et al. Doxorubicin downregulates cell surface B7-H1 expression and upregulates its nuclear expression in breast cancer cells: role of B7-H1 as an anti-apoptotic molecule. Breast Cancer Res. 2010; 12:R48.

19. Zhang P, Su D M, Liang M, Fu J. Chemopreventive agents induce programmed death-1-ligand 1 (PD-L1) surface expression in breast cancer cells and promote PD-L1-mediated T cell apoptosis. Molecular immunology. 2008; 45:1470-6.

20. Deng L, Liang H, Burnette B, Beckett M, Darga T, Weichselbaum RR, et al. Irradiation and anti-PD-Ll treatment synergistically promote antitumor immunity in mice. J Clin Invest. 2014; 124:687-95.

21. Ghiotto M, Gauthier L, Serriari N, Pastor S, Truneh A, Nunes J A, et al. PD-L1 and PD-L2 differ in their molecular mechanisms of interaction with PD-1. International immunology. 2010; 22:651-60.

22. Brom M, Joosten L, Oyen W J, Gotthardt M, Boerman O C. Improved labelling of DTPA- and DOTA-conjugated peptides and antibodies with ¹¹¹In in HEPES and MES buffer. EJNMMI Res. 2012; 2:4.

23. van der Have F, Vastenhouw B, Ramakers R M, Branderhorst W, Krah JO, Ji C, et al. U-SPECT-II: An Ultra-High-Resolution Device for Molecular Small-Animal Imaging. J Nucl Med. 2009; 50:599-605.

24. Heldin C H, Rubin K, Pietras K, Ostman A. High interstitial fluid pressure—an obstacle in cancer therapy. Nat Rev Cancer. 2004; 4:806-13.

25. Jain R K. Transport of molecules, particles, and cells in solid tumors. Annu Rev Biomed Eng. 1999; 1:241-63.

26. Press O W, Shan D, Howell-Clark J, Eary J, Appelbaum F R, Matthews D, et al. Comparative metabolism and retention of iodine-125, yttrium-90, and indium-111 radioimmunoconjugates by cancer cells. Cancer Res. 1996; 56:2123-9.

27. Fang J, Nakamura H, Maeda H. The EPR effect: Unique features of tumor blood vessels for drug delivery, factors involved, and limitations and augmentation of the effect. Advanced drug delivery reviews. 2011; 63:136-51.

28. Dijkers E C, Kosterink J G, Rademaker A P, Perk L R, van Dongen G A, Bart J, et al. Development and characterization of clinical-grade 89Zr-trastuzumab for HER2/neu immunoPET imaging. J Nucl Med. 2009; 50:974-81.

29. Heskamp S, van Laarhoven H W, Molkenboer-Kuenen J D, Franssen G M, Versleijen-Jonkers Y M, Oyen W J, et al. ImmunoSPECT and immunoPET of IGF-1R expression with the radiolabeled antibody R1507 in a triple-negative breast cancer model. J Nucl Med. 2010; 51:1565-72.

30. Marquez B V, Ikotun O F, Zheleznyak A, Wright B, Hari-Raj A, Pierce R A, et al. Evaluation of (89)Zr-pertuzumab in Breast cancer xenografts. Molecular pharmaceutics. 2014; 11:3988-95.

31. Heskamp S, Boerman O C, Molkenboer-Kuenen J D, Oyen W J, van der Graaf W T, van Laarhoven H W. Bevacizumab reduces tumor targeting of anti-epidermal growth factor and anti-insulin-like growth factor 1 receptor antibodies. Int J Cancer. 2013.

32. Van der Auwera I, Van Laere S J, Van den Eynden G G, Benoy I, van Dam P, Colpaert C G, et al. Increased angiogenesis and lymphangiogenesis in inflammatory versus noninflammatory breast cancer by real-time reverse transcriptase-PCR gene expression quantification. Clin Cancer Res. 2004; 10:7965-71.

33. Bieche I, Lerebours F, Tozlu S, Espie M, Marty M, Lidereau R. Molecular profiling of inflammatory breast cancer: identification of a poor-prognosis gene expression signature. Clin Cancer Res. 2004; 10:6789-95.

34. Dong H, Zhu G, Tamada K, Chen L. B7-H1, a third member of the B7 family, co-stimulates T-cell proliferation and interleukin-10 secretion. Nature medicine. 1999; 5:1365-9.

35. Freeman G J, Long A J, Iwai Y, Bourque K, Chernova T, Nishimura H, et al. Engagement of the PD-1 immunoinhibitory receptor by a novel B7 family member leads to negative regulation of lymphocyte activation. J Exp Med. 2000; 192:1027-34.

36. Hodi F S, Powles T, Cassier P, Kowanetz M, Herbst R S, Soria J C, et al. MPDL3280A (anti-PDL1): Clinical activity, safety and biomarkers of an engineered PD-L1 antibody in patients with locally advanced or metastatic tumors. European Journal of Cancer. 2013; 49:S184-S.

1-10. (canceled)

11. A method of treating a tumor in a patient, comprising the step of administering to said patient a therapeutically effective amount of an anti-PD-L1 immunotoxin, wherein said immunotoxin comprises an anti-PD-L1 antibody linked to a toxin.

12. The method according to claim 11, wherein said anti-PD-L1 antibody is a monoclonal antibody.

13. The method according to claim 11, wherein said toxin comprises a Ribosome Inactivating Protein.

14. The method according to claim 11, wherein said toxin comprises a Ribosome Inactivating Protein chosen from saporin, ricin, abrin, gelonin, Pseudomonas exotoxin trichosanthin, luffin, agglutinin and diphtheria toxin.

15. The method according to claim 11, wherein said toxin is a chemical drug chosen from:
modeccin, mitogellin, chiortetracyclgne, mertansine, monomethyl auristatin E, monomethyl auristatin F and enediynes, especially calicheamicins and their related esperamicins;
anticancer agents, preferably chosen from combrestatin, colchicine, actinomycine, duocarmycins and their synthetic analogues, fludarabine, gemcitabine, capecitabine, methotrexate, taxol, taxotere, mercaptopurine, thioguanine, hydroxyurea, cytarabine, cyclophosphamide, ifosfamide, platinum complexes, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epirubicin, 5-fluorouracil, taxanes, leucovorin, levamisole, irinotecan, estramustine, etoposide, nitrogen mustards, BCNU, nitrosoureas, vinca alkaloids, imatinib mesylate, hexamethylenediamine, topotecan, kinase inhibitors, phosphatase inhibitors, ATPase inhibitors, protease inhibitors, inhibitors of herbimycin A, genistein, erbstatin, and lavendustin A; and
radioisotopes, preferably chosen from ²¹¹At, ¹³¹I, ¹²⁵I, ¹⁸⁶Re, ¹⁸⁸Re, ¹⁵³Sm, P³², ⁹⁰Y, ¹⁷⁷Lu, ⁶⁷Cu, ⁴⁷Sc, ²¹²Bi, ²¹³Bi, ²²⁶Th, ¹¹¹In and ⁶⁷Ga.

16. The method according to claim 11, wherein said tumor is a solid tumor selected in the group consisting of prostate cancer, pancreatic cancer, breast cancer, melanoma, B cell lymphoma, brain cancer, bladder cancer, colon cancer, intestinal cancer, lung cancer, stomach cancer, cervical cancer, ovarian cancer, liver cancer, skin cancer, colorectal cancer, endometrial carcinoma, salivary gland carcinoma, kidney cancer, thyroid cancer, various types of head and neck cancer.

17. Pharmaceutical composition comprising an anti-PD-L1 immunotoxin wherein said anti-PD-L1 immunotoxin comprises an anti-PD-L1 antibody linked to a toxin.
